(19) United States
(12) Patent Application Publication
(10) Pub. No.: US 2006/0252803 A1
(43) Pub. Date: Nov. 9, 2006

(54) Hydrogenation of precursors to thiazolidinedione antihyperglycemics

(76) Inventors: Ben-Zion Dolitzky, Petach Tiqva (IL); Michael Pesachovich, Givat-Shmuel (IL)

Correspondence Address: KENYON & KENYON LLP, ONE BROADWAY, NEW YORK, NY, 10004, US

(21) Appl. No.: 11/341,006

(22) Filed: Jan. 26, 2006

Related U.S. Application Data
(63) Continuation-in-part of application No. 10/324,928, filed on Dec. 20, 2002, now Pat. No. 6,992,191.
(60) Provisional application No. 60/342,437, filed on Dec. 20, 2001.

(51) Int. Cl. A61K 31/4439; C07D 417/02

(52) U.S. Cl. 514/340; 546/269.7

(57) ABSTRACT

Provided is pioglitazone having a low level of impurities, especially a low level of the precursor PIE. Also provided is a method for making pioglitazone having a low level of impurities.

### RELATED APPLICATIONS

This application is a continuation-in-part of U.S. patent application Ser. No. 10/324,928, filed Dec. 20, 2002, which claims the benefit of U.S. Provisional Application No. 60/342,437, filed Dec. 20, 2001, the contents of all of which are incorporated herein by reference.

### FIELD OF THE INVENTION

The present invention relates to a method of making thiazolidinedione antihyperglycemics that includes the step of catalytic hydrogenation of a penultimate thiazolidinedione precursor.

### BACKGROUND OF THE INVENTION

Diabetes is a disorder of metabolism in which either the pancreas produces too little or no insulin, or the body cells do not respond to the insulin that is produced. In type I diabetes, the pancreas does not produce any insulin. In type II diabetes, also known as adult onset diabetes, there are two potential problems: the pancreas produces too little insulin, or the body cells do not respond to the insulin that is produced. In either scenario, the glucose cannot efficiently move from the blood to the cells, which leads to a buildup of glucose in the blood and an overflow into the urine. As a result, the body loses its main source of fuel. Administering insulin or oral antihyperglycemic agents allows the glucose to enter the cells more efficiently, thus providing a source of fuel.

Thiazolidinedione antihyperglycemics(benzylidenethiazolidinedione antihyperglycemics) are a class of drugs, useful in treating type II diabetes and other disorders relating to insulin resistance, that share a 5-(4-alkoxyphenyl)methyl-2,4-thiazolidinedione (I) pharmacophore.

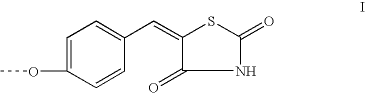

Pioglitazone is an oral thiazolidinedione antihyperglycemic agent that acts primarily by decreasing insulin resistance. Pharmacological studies indicate that pioglitazone improves sensitivity to insulin in muscle and adipose tissue and inhibits hepatic gluconeogenesis. Pioglitazone improves glucose resistance while reducing circulating insulin levels.

Pioglitazone, as its hydrochloride, is currently marketed as ACTOS®. Pioglitazone hydrochloride has the chemical name [(±)5-[[4-[2-(5-ethyl-2-pyridinyl)ethoxy]phenyl]methyl]-2,4-]thiazolidinedione monohydrochloride. (CAS Registry No. 111025-46-8). The chemical structure of pioglitazone is shown as structure II.

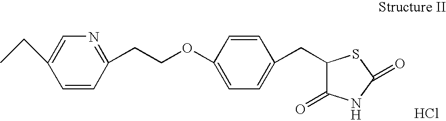

U.S. Pat. No. 5,952,509, incorporated herein by reference, discloses methods for the synthesis of pioglitazone.

Rosiglitazone, 5-[4-[2-[N-methyl-N-(pyridin-2-yl)aminoethoxy]phenyl]methyl-2,4-thiazolidinedione, and troglitazone, 5-[4-[(6-hydroxy-2,5,7,8-tetramethylchroman-2-yl)methoxy]benzyl]-2,4-thiazolidinedione, are also a thiazolidinedione antihyperglycemics useful in treating type II diabetes and other disorders relating to insulin resistance. Rosiglitazone is marketed under the trade name Avandia®. Troglitazone has been marketed under the trade name Prelay®.

Methods for making pioglitazone, rosiglitazone, and troglitazone may proceed via a thiazolidinedione precursor having an exocyclic carbon-carbon double bond at the 5 position of a thiazolidinedione ring. The method of making pioglitazone disclosed in U.S. Pat. No. 5,952,509 is such a method. In such methods, the carbon-carbon double bond must be hydrogenated to a carbon-carbon single bond to form the thiazolidinedione antihyperglycemic. Catalytic hydrogenation over a supported catalyst, a method generally well known in the art, has been used to this end.

Synthesis of rosiglitazone via a thiazolidinedione precursor is disclosed in, for example, U.S. Pat. No. 5,002,953 (the '953 patent). Synthesis of troglitazone via a thiazolidinedione precursor is disclosed in J. Cossy et al., A Short Synthesis of Troglitazone: An Antidiabetic Drug for Treating Insulin Resistance, 9 Bioorganic and Medicinal Chemistry Letters, 3439-3440 (1999).

When the thiazolidinedione precursor is a solid, which is usually the case, a solvent must be used in the hydrogenation step. Hydrogenation of the thiazolidinedione pioglitazone precursors in solvents such as dioxane and particularly DMF has been reported. Large quantities (up to 20 volumes) of such solvents are required. When these solvents may be used, higher pressures (e.g. 50-100 atm) and a large amount of catalyst (ratio of weight of catalyst to weight of precursor of 1 to 3) are required. Even with such large amounts of catalyst, longer reaction times, e.g. ≧72 hr in some cases, are required to obtain only fair yields, e.g. 35-40%.

Generally, side products, by-products, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time”, relative to an internal reference marker.

The retention time can vary about a mean value based upon the condition of the instrumentation, as well as many other factors. To mitigate the effects such variations have upon accurate identification of an impurity, practitioners use the “relative retention time” (“RRT”) to identify impurities (Strobel p. 922). The RRT of an impurity is its retention time divided by the retention time of a reference marker. It may be advantageous to select a compound other than the API that is added to, or present in, the mixture in an amount sufficiently large to be detectable and sufficiently low as not to saturate the column, and to use that compound as the reference marker for determination of the RRT.

### SUMMARY OF THE INVENTION

The present invention provides, i.a., a method for making thiazolidinedione antihyperglycemics from a thiazolidinedione precursor that includes the step of catalytically hydrogenating a thiazolidinedione precursor having an exocyclic double bond at the 5 position of the thiazolidine ring in a high capacity solvent.

In one aspect, the present invention relates to a method of hydrogenating a thiazolidinedione precursor, especially a thiazolidinedione precursor for pioglitazone, rosiglitazone, or troglitazone, including the steps of: providing a solution of the thiazolidinedione precursor in a high capacity solvent, especially formic acid, combining the solution with a supported metal hydrogenation catalyst, exposing the combination of solution and hydrogenation catalyst to hydrogen gas, and isolating hydrogenated precursor.

In another aspect, the present invention relates to a method of hydrogenating a penultimate thiazolidinedione precursor, especially a penultimate thiazolidinedione precursor of pioglitazone, rosiglitazone, or troglitazone including the steps of: providing a solution of the penultimate thiazolidinedione precursor in a high capacity solvent, especially formic acid, wherein the concentration of the solution is at least about 0.25 g/mL, especially at least about 0.5 g/mL; combining the solution with a supported metal hydrogenation catalyst, especially one in which the metal is selected from platinum, palladium, ruthenium, rhodium, osmium, and iridium; and exposing the combination of solution and hydrogenation catalyst to hydrogen gas, or without hydrogen gas.

In still another aspect, the present invention relates to a method of hydrogenating a penultimate thiazolidinedione precursor, especially a penultimate thiazolidinedione precursor for pioglitazone, rosiglitazone, or troglitazone including the steps of: providing a solution of the penultimate thiazolidinedione precursor in a high capacity solvent, especially formic acid, wherein the concentration of the solution is at least about 0.25 g/mL, especially at least about 0.5 g/mL; combining the solution with a supported metal hydrogenation catalyst selected from platinum, ruthenium, rhodium, osmium, iridium, and, especially, palladium, whereby the ratio of the weight of metal to the weight of precursor is about 0.03:1 or less, especially about 0.02:1; exposing the combination of solution and hydrogenation catalyst to hydrogen gas at a pressure between about 1 and about 10 Atm and a temperature between about 40° C. and about 100° C., and isolating the thiazolidinedione antihyperglycemic.

In still another aspect, the present invention provides a method for making pioglitazone including the step of catalytically hydrogenating 5-[4-[2-[5ethylpyridin-2-yl]ethoxy]phenyl]methenyl-2,4-thiazolidinedione in solution in a high capacity solvent, especially formic acid, using a supported metal catalyst wherein the metal is selected from platinum, ruthenium, rhodium, osmium, iridium, and, especially, palladium and the amount of catalyst is such that the ratio of the weight of the metal to the weight of precursor is less than about 0.03:1, especially 0.02:1 or less; exposing the combination of solution and hydrogenation catalyst to hydrogen gas at a pressure between about 1 and 10 Atm. and a temperature between about 40° C. and about 100° C., especially 75° to 85° C.; and isolating pioglitazone.

In still a further aspect, the present invention relates to a method of making pure pioglitazone including the step of catalytically hydrogenating 5-[4-[2-[5ethylpyridin-2-yl]ethoxy]phenyl]methenyl-2,4-thiazolidinedione in solution in a high capacity solvent, especially formic acid, using a supported metal catalyst wherein the metal is selected from platinum, ruthenium, rhodium, osmium, iridium, and, especially, palladium and the amount of catalyst is such that the ratio of the weight of the metal to the weight of precursor is less than about 0.03:1, especially 0.02:1 or less; exposing the combination of solution and hydrogenation catalyst to hydrogen gas at a pressure between about 1 and 10 Atm. and a temperature between about 40° C. and about 100° C.; isolating the product of the catalytic hydrogenation and slurrying the isolated product in a slurry solvent selected from acetone, methanol, ethanol and isopropanol; and isolating pure pioglitazone.

In still a further aspect, the present invention relates to a method of making rosiglitazone including the step of catalytically hydrogenating 5-[4-[2-[N-methyl-N-(pyridin-2-yl)aminoethoxy]phenyl]methenyl-2,4-thiazolidinedione in solution in a high capacity solvent, especially formic acid, using a supported metal catalyst wherein the metal is selected from platinum, ruthenium, rhodium, osmium, iridium, and, especially, palladium and the amount of catalyst is such that the ratio of the weight of the metal to the weight of precursor is less than about 0.03:1, especially 0.02:1 or less; exposing the combination of solution and hydrogenation catalyst to hydrogen gas at a pressure between about 1 and 10 Atm. and a temperature between about 40° C. and about 100° C.; and isolating rosiglitazone.

In still a further aspect, the present invention relates to a method of making pure rosiglitazone including the step of catalytically hydrogenating 5-[4-[2-[N-methyl-N-(pyridin-2-yl)aminoethoxy]phenyl]methenyl-2,4-thiazolidinedione in solution in a high capacity solvent, especially formic acid, using a supported metal catalyst wherein the metal is selected from platinum, ruthenium, rhodium, osmium, iridium, and, especially, palladium and the amount of catalyst is such that the ratio of the weight of the metal to the weight of precursor is less than about 0.03:1, especially 0.02:1 or less; exposing the combination of solution and hydrogenation catalyst to hydrogen gas at a pressure between about 1 and 10 Atm and a temperature between about 40° C. and about 100° C.; isolating the product of the catalytic hydrogenation and slurrying the isolated product in a slurry solvent selected from acetone, methanol, ethanol and isopropanol; and isolating pure rosiglitazone.

In still a further aspect, the present invention relates to a method of making troglitazone including the step of catalytically hydrogenating 5-[4-[(6-hydroxy-2,5,7,8-tetramethylchroman-2-yl)methoxy]phenyl]methenyl-2,4-thiazolidinedione in solution in a high capacity solvent, especially formic acid, using a supported metal catalyst wherein the metal is selected from platinum, ruthenium, rhodium, osmium, iridium, and, especially, palladium and the amount of catalyst is such that the ratio of the weight of the metal to the weight of precursor is less than about 0.03:1, especially 0.02:1 or less; exposing the combination of solution and hydrogenation catalyst to hydrogen gas at a pressure between about 1 and 10 Atm and a temperature between about 40° C. and about 100° C.; and isolating troglitazone.

In still a further aspect, the present invention relates to a method of making pure troglitazone including the step of catalytically hydrogenating 5-[4-[(6-hydroxy-2,5,7,8-tetramethylchroman-2-yl)methoxy]phenyl]methenyl-2,4-thiazolidinedione in solution in a high capacity solvent, especially formic acid, using a supported metal catalyst wherein the metal is selected from platinum, ruthenium, rhodium, osmium, iridium, and, especially, palladium and the amount of catalyst is such that the ratio of the weight of the metal to the weight of precursor is less than about 0.03:1, especially 0.02:1 or less; exposing the combination of solution and hydrogenation catalyst to hydrogen gas at a pressure between about 1 to 10 Atm. and a temperature between about 40° C. and about 100° C.; isolating the product of the catalytic hydrogenation and slurrying the isolated product in a slurry solvent selected from acetone, methanol, ethanol and isopropanol; and isolating pure rosiglitazone.

In a further aspect, the present invention relates to pioglitazone containing less than about 0.14% area by HPLC of the impurity having RRT of 0.64, as determined by the hereinbelow-described HPLC method. Preferably, the pioglitazone contains less than about 0.02% area by HPLC of the impurity at RRT 0.64.

In still a further aspect, the present invention relates to a method of making pioglitazone containing less than about 0.14% by area by HPLC of the impurity having RRT of 0.64, as determined by the hereinbelow-described HPLC method, including the steps of: providing a solution of PIE in a high capacity solvent solvent, especially formic acid; combining the solution with a supported metal hydrogenation catalyst in a reactor; heating the combination to a temperature of about 40° C. to about 100° C.; separating the supported metal catalyst from the solution; combining the solution with a crystallization solvent selected from the group consisting of acetone and a lower aliphatic alcohol; and recovering the solid pioglitazone having less than about 0.14% are by HPLC of the impurity at RRT 0.64.

Preferably, the pioglitazone obtained by the above process contains less than about 0.02% area by HPLC of the impurity having RRT 64, as determined by the hereinbelow-described HPLC method.

### DETAILED DESCRIPTION OF THE INVENTION

The present invention provides a method for making a thiazolidinedione antihyperglycemic via a thiazolidinedione precursor having an exocyclic double bond at the 5 position of the thiazolidinedione ring thereof, which method includes the step of catalytic hydrogenation with a supported metal catalyst in which less catalyst is required (as little as 0.2 gram of catalyst per gram of precursor) and in which good yields (e.g. ≧85% ) can be realized in reaction times of 30 hr or less.

The present invention provides a method for making pioglitazone, rosiglitazone, and troglitazone from respective thiazolidinedione precursors that includes the step of catalytically hydrogenating the thiazolidinedione precursor having an exocyclic carbon-carbon double bond at the 5 position of the thiazolidine ring, wherein the hydrogenation is carried-out in a high capacity solvent.

A thiazolidinedione precursor is a compound that is an intermediate in a process for making a thiazolidinedione antihyperglycemic, such as the process disclosed in U.S. Pat. No. 5,952,509 incorporated herein by reference, and that has a thiazolidinedione moiety. Thiazolidinedione pioglitazone precursors useful in the practice of the present invention have an exocyclic double bond at the 5 position of the thiazolidinedione moiety as illustrated below.

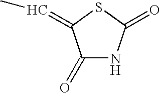

Preferred thiazolidinedione pioglitazone precursors are penultimate thiazolidinedione precursors. A penultimate thiazolidinedione precursor differs structurally from the thiazolidinedione antihyperglycemic itself in that the penultimate thiazolidinedione precursor has an exocyclic double bond at the 5-position of the thiazolidinedione moiety. A penultimate thiazolidinedione precursor may also have protected functional groups groups (i.e. protected hydroxyl groups). Hydrogenation of this exocyclic double bond, and removal of protecting groups if any, yields the thiazolidinedione antihyperglycemic, which is isolated from the reaction mixture. The compound 5-[4-[2-[5-ethylpyridin-2-yl]ethoxy]phenyl]methenyltiazolidine-2,4-dione (hereafter “PIE”) is an example of a penultimate thiazolidinedione precursor for pioglitazone.

Thus, hydrogenation of the exocyclic double bond of the penultimate thiazolidinedione pioglitazone precursor PIE affords pioglitazone as illustrated in reaction I below in which the supported metal hydrogenation catalyst is palladium-on-carbon (Pd/C) catalyst.

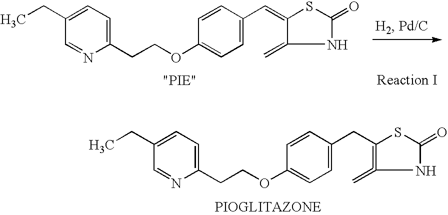

Synthesis of PIE is taught, for example, in U.S. Pat. No. 5,952,509.

Hydrogenation of the penultimate thiazolidinedione precursor 5-[4-[2-[N-methyl-N-(pyridin-2-yl)aminoethoxy]phenyl]methenyl-2,4-thiazolidinedione affords rosiglitazone. Synthesis of 5-[4-[2-[N-methyl-N-(pyridin-2-yl)aminoethoxy]phenyl]methenyl-2,4-thiazolidinedione is disclosed in, for example, the '953 patent. Likewise, hydrogenation of the penultimate thiazolidinedione precursor 5-[4-[(6-hydroxy-2,5,7,8-tetramethylchroman-2-yl)methoxy]phenyl]methenyl-2,4-thiazolidinedione, or hydroxy group protected derivatives thereof, affords troglitazone,. See J. Cossy et al., supra.

The hydrogenation step of the present invention is catalytic hydrogenation over a supported metal hydrogenation catalyst. Supported metal hydrogenation catalysts are well known in the art and have a metal deposited, absorbed, or coated on or in a solid support. Examples of metals that can be used include platinum, palladium, ruthenium, rhodium, osmium, and iridium. Many solid supports are known in the art. Particulate carbon is a well-known useful solid support. Supported metal hydrogenation catalysts are described in, for example, Shigeo Nishimura, Handbook of Heterogeneous Catalytic Hydrogenation for Organic Synthesis, Chpt. 1, (2001). Palladium catalyst supported on carbon (Pd/C catalyst) is a preferred supported metal hydrogenation catalyst for use in the present invention. An example of a preferred Pd/C catalyst useful in the practice of the present invention is 87 L powder catalyst (10% Pd by weight) available from Johnson Matthey, West Depford, N.J.

In the practice of the present invention, the catalytic hydrogenation of the exocyclic double bond of the thiazolidinedione precursor is carried-out in a high-capacity solvent. A high capacity solvent is one in which one gram (1 g) of thiazolidinedione pioglitazone precursor dissolves in about 5 milliliters (5 mL) or less of solvent. Preferred high capacity solvents are those in which 1 g of precursor dissolves in 4 mL or less of solvent at a temperature between about 25° C. and about 45° C. Formic acid is particularly preferred high capacity solvent in the practice of the present invention. When used as the high capacity solvent, the formic acid can have up to about 15% by weight water.

In the practice of the present invention, the weight of supported metal hydrogenation catalyst used is preferably such that the ratio of the weight of metal to the weight of precursor to be hydrogenated is about 0.05:1 or less, preferably 0.03:1 or less. Most preferably, the amount of catalyst is such that the ratio of the weight of metal to the weight of precursor is about 0.02:1 or less. The weight of the metal is calculated by multiplying the weight of the supported metal catalyst by the percent catalyst loading expressed as a decimal. Thus, if the weight ratio of 10% loaded supported metal catalyst to precursor is 0.2:1; the ration of the weight of metal to the weight of precursor is 0.02:1.

The catalytic hydrogenation of thiazolidinedione pioglitazone precursor is carried-out in conventional equipment well known in the art. For example, in an autoclave. The autoclave can be equipped with a stirrer or it can be of the shaker-type. The hydrogen pressure to which the solution is exposed during hydrogenation is not critical to realizing the benefits of the present invention. In particular embodiments, hydrogen gas is not used. Typically, the solution is exposed to a hydrogen pressure between about 1 and about 10 Atm, preferably about 2 to about 5 Atm.

In a particular embodiment in which formic acid is the high capacity solvent, hydrogenation is effected without exposing the solution of thiazolidinedione precursor, preferably penultimate precursor, to hydrogen gas. In this embodiment, a solution of the thiazolidinedione precursor in formic acid is combined with supported metal hydrogenation catalyst and heated at about 40° C. to about 100° C. The amounts of solvent and catalyst are the same as in other embodiments.

In a preferred embodiment, the hydrogenation reactor (e.g. autoclave) is purged at least once, preferably at regular intervals (e.g. 30 min.), during the hydrogenation reaction. In a purging step, gas supply to the reactor is closed off, the reactor is vented to the atmosphere, and gas supply is re-established to repressurize the reactor with hydrogen gas.

The skilled artisan will recognize that any operation or procedure that allows for refreshment of the atmosphere in the reactor is a purging step and such operations that allow refreshment of the atmosphere in the reactor are within the scope of the invention.

The temperature at which the catalytic hydrogenation in a high capacity solvent of the present invention is carried-out is not critical and will be influenced by, among other things, practical considerations such as reactor throughput and operational safety. Typically, the temperature will be between about 40° C. and 100° C., preferably between about 70° C. and about 90° C., but temperatures≧100° C. can be used without sacrificing the benefits of the present invention.

The time of hydrogenation is not critical. However, it is an advantage of the present invention over prior art methods that, parameters such as H₂ pressure, catalyst dosage (g catalyst per g precursor), catalyst loading (percent of catalyst not consisting of carbon or other support), and catalyst surface area (such as can be measured by, for example, nitrogen absorption) being equal, the present invention allows for shorter hydrogenation times (time to completion of reaction), without sacrifice in conversion, yield, or purity. Compared to results obtained practicing methods of the prior art, higher degrees of reaction completion and higher yields of pioglitazone are obtained in less hydrogenation time when the method of the present invention is used. The skilled artisan will know to judge completion of the reaction by, for example, noting a cessation of hydrogen uptake, or by sampling the contents of the reactor using known techniques and analyzing the sample using, for example, gas chromatography.

In the practice of preferred embodiments of the catalytic hydrogenation in a high capacity solvent, a slurry is obtained wherein the hydrogenation product is in solution in the high capacity solvent at the completion of hydrogenation. The product can be recovered by, for example, adding a non-solvent to the solution or by concentrating the solution, especially under vacuum, whereby a suspension or slurry forms from which the product can be isolated. In this and other embodiments of the present invention, isolation can be by any means known in the art, for example filtration (gravity or suction) or centrifugation, to mention just two.

The conversions realized in the method of the present invention are at least about 99% and the hydrogenation product contains less than 0.1 area-% residual thiazolidinedione precursor, typically 0.05 area-% or less. In a preferred embodiment in which PIE is the penultimate thiazolidinedione antihyperglycemic precursor, the final pioglitazone has less than 0,05 area-% PIE and, in particularly preferred embodiments about or less than 0.02 area-% PIE as determined by the hereinbelow described HPLC method.

In a further embodiment, the present invention provides a recovery process for work-up of the thiazolidinedione antihyperglycemic product of hydrogenation of a penultimate thiazolidinedione precursor to afford pure thiazolidinedione antihyperglycemic. The recovery process includes the steps of separating catalyst from the solution at the completion of the hydrogenation, adding a crystallization solvent to the solution from which catalyst was separated, cooling the combination whereby a solid precipitate of thiazolidinedione antihyperglycemic forms, and isolating the thiazolidinedione antihyperglycemic.

In preferred embodiments, the solution from which catalyst has been separated is concentrated before being combined with crystallization solvent. Any degree of concentration can improve recovery. Typically, the solution will be concentrated to about 60% to about 40% of its initial weight.

Acetone and lower alkyl alcohols can be used as crystallization solvents. Lower alkyl alcohols useful in the practice of the present invention have the formula ROH, wherein R is a linear or branched alkyl group having up to 6 carbon atoms. Methanol, ethanol, and isopropanol are preferred lower alkyl alcohols. Ethanol is a particularly preferred lower alkyl alcohol for use in the practice of the present invention. The skilled artisan will know to adjust, by routine optimization, the amount of crystallization solvent according to, for example, the concentration of the solution with which the crystallization solvent is combined. If the solution is not concentrated, the amount of crystallization solvent will typically be about 7 to about 12 timed the volume of solution.

The thiazolidinedione antihyperglycemic isolated from the recovery process is pure thiazolidinedione antihyperglycemic. Pure denotes that the antihyperglycemic has a purity of at least about 99.7%, expressed as area percent, as determined by high-pressure liquid chromatography (HPLC) according to the method described below.

In another embodiment, the present invention provides pioglitazone containing less than about 0.14% area by HPLC of the impurity at RRT 0.64. Preferably, the pioglitazone of the present invention contains less than about 0.02% area by HPLC of the impurity at RRT 0.64.

As used herein in connection with the a thiazolidinedione antihyperglycemic (e.g. pioglitazone) or impurities therein, purity or %-impurity refers to area-% determined by the hereinbelow-described HPLC method.

Area percent (area-%) refers to one hundred times the quotient of the area of the peak in an HPLC chromatogram resulting from elution of the species in question (e.g. PIE), monitored by, for example, a UV detector as known in the art, upon the total sum of the areas of all peaks in the HPLC chromatogram. Area percent can be expressed mathematically as:

$$100 \times ( Ai / {\sum }_{i} Ai )$$

Where “i” is the total number of peaks in the HPLC chromatogram.

The RRT values expressed herein are specific to the HPLC conditions disclosed herein.

Purity (area-% purity) is determined by HPLC using a 250×4.6 mm column packed with YMC ODS AQ (5 μ) at 40° C. and eluent flow rate of 1.0 ml/min. Detection is with a UV detector operating at 220 nm. Elution is by linear gradient elution according to the following program:

| Elution Time | | |
|---|---|---|
| (min) | % Eluent A | % Eluent B |
| 0 | 100 | 0 |
| 3 | 100 | 0 |
| 33 | 20 | 80; |

wherein eluent A is 60% 0.01M aqueous trifluoroacetic acid (adjusted to pH 2.5 with 1N KOH_{aq}) and 40% methanol and wherein eluent B is 30% 0.01M aqueous trifluoroacetic acid (adjusted to pH 2.5 with 1N KOH_{aq}). The nominal injection volume is 20 μL.

The detection limit for impurities (species other than the tiazolidinedione antihyperglycemic) of the HPLC method is 0.02 area-%.

Purity (area-% purity) for the impurity at RRT 0.64 was determined by HPLC using a 250×4.6 mm column packed with YMC ODS AQ (5 μ) at 40° C. and eluent flow rate of 1.0 ml/min. Detection is with a UV detector operating at 269 nm. Elution is by linear gradient elution according to the following program:

| Elution Time | | |
|---|---|---|
| (min) | % Eluent A | % Eluent B |
| 0 | 100 | 0 |
| 10 | 100 | 0 |
| 35 | 63 | 37 |
| 50 | 63 | 37 |

wherein eluent A is 57% 0.02M H₃PO₄ (adjusted to pH 2.7 with 5N KOH_{aq}) and 43% methanol and wherein eluent B is methanol. The nominal injection volume is 50 μL. Equilibrium time: 10 minutes.

Diluent, for use for example in introducing sample to the HPLC, was prepared by dissolving in either a) 60% methanol and diluting to 100% with 0.02M H3PO4 adjusted to pH 2.7 with 5N KOH, or b) their mixture.

The detection limit for impurities (species other than the tiazolidinedione antihyperglycemic) of the HPLC method is 0.02 area-%.

The present invention also relates to a method of making pioglitazone containing less than about 0.14% are by HPLC of the impurity having RRT 0.64, comprising the steps of: providing a solution of PIE in a high capacity solvent solvent, especially formic acid; combining the solution with a supported metal hydrogenation catalyst in a reactor, wherein the supported metal hydrogenation catalyst comprises a metal selected from the group consisting of platinum, palladium, ruthenium, rhodium, osmium, and iridium; heating the combination to a temperature of about 40° C. to about 100° C.; especially about 80° C.; separating supported metal catalyst from the solution; optionally concentrating the solution, especially at reduced pressure, combining the optionally-concentrated solution from which catalyst had been separated with a crystallization solvent selected from the group consisting of acetone and a lower aliphatic alcohol, especially ethanol; and recovering the solid pioglitazone having less than about 0.14% are by HPLC of the impurity at RRT 0.64.

Preferably, the pioglitazone obtained by the above process contains less than about 0.02% area by HPLC of the impurity at RRT 0.64.

In a further embodiment, the present invention provides pharmaceutical compositions (formulations) comprising pioglitazone containing less than about 0.14% area by HPLC of the impurity at RRT 0.64, preferably less than about 0.02% area by HPLC of the impurity at RRT 0.64.

Pharmaceutical compositions of the present invention contain solid pioglitazone obtained by the method of the present invention in any of its embodiments. In addition to the active ingredient(s), the pharmaceutical formulations of the present invention can and typically do contain one or more pharmaceutically acceptable excipients. Excipients are added to the formulation for a variety of purposes. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc, to mention just a few.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the pioglitazone and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxyl toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid pharmaceutical compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms such as tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient, pioglitazone, and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition can be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The present invention is further illustrated by the following non-limiting Examples 1 to 4. Examples 5 and 6 are comparative examples showing the results obtained when following a method from the prior art that does not use a high capacity solvent.

### EXAMPLES

### Example 1

One gram of PIE is charged to a test tube. One milliliter of formic acid is added to the test tube. The test tube is agitated by hand in a bath maintained at 45° C. A clear solution forms, showing that formic acid is a high capacity solvent.

### Example 2

Ten grams of Pd/C catalyst (Johnson Matthey 87L, 10% Pd), 200 ml formic acid, and 50 g PIE were charged to a laboratory autoclave. The autoclave was closed, charged with H₂, and heated to 60° C. The H₂ pressure was adjusted to 2 Atm. The contents of the autoclave were maintained at 60° C. under 2 Atm H₂ pressure for 30 hours.

Heating was stopped, the pressure released, and the autoclave opened while the contents, a slurry, were still warm. The slurry was filtered warm and washed with two 20 ml aliquots of formic acid. Analysis showed that 39% of PIE had been converted to pioglitazone; only 0.24% of the starting PIE remained unreacted.

One and eight-tenths liter of acetone were added to the recovered solution and the resulting solution was allowed to stand for 5 hrs, during which time the product crystallized from solution. The slurry was filtered and washed with 20 ml of a 9:1 mixture of acetone and formic acid. The recovered product was dried to give 42 g (yield 84%) pioglitazone having a purity of 39.7% (HPLC).

### Example 3

Ten grams of Pd/C catalyst (Johnson Matthey 87 L, 10% Pd), 200 ml formic acid, and 50 g PIE were charged to a laboratory autoclave. The autoclave was closed, charged with H₂, and heated to 60° C. The H₂ pressure was adjusted to 6 Atm. The contents of the autoclave were maintained at 60 C. under 6 Atm H₂ pressure for 30 hours.

Heating was stopped, the pressure released, and the autoclave opened while the contents, a slurry, were still warm. The slurry was filtered warm and washed with two 20 ml aliquots of formic acid. Analysis showed that ≧99% of PIE had been converted to pioglitazone; only 0.24% of the starting PIE remained unreacted.

One and eight-tenths liter of acetone were added to the recovered solution and the resulting solution was allowed to stand for 5 hrs, during which time the product crystallized from solution. The slurry was filtered and washed with 20 ml of a 9:1 mixture of acetone and formic acid. The recovered product was dried to give 42 g (yield 84%) pioglitazone having a purity of ≧99.7% (HPLC).

### Example 4

PIE (50 kg.) was dissolved in formic acid (500 kg,). Supported metal catalyst (40 kg. of 10% Pd on carbon, KF=50%) was added and the suspension was heated to 80° C. and pressureized to 2 Atm with hydrogen. The reactor was purged at 30 minute intervals throughout the hydrogenation.

After 20 hours, the suspension was cooled to room temperature and the catalyst separated by filtration. The solution was concentrated to 80 kg. Ethanol (632 kg) was added to the solution at 75° C. and the resulting mixture was gradually cooled to <13° C. The precipitate formed was isolated by filtration and washed with ethanol. Yield: 30 kg after drying. Level of impurity at RRT of 0.64:<0.02%.

The just-recited procedure was repeated three times and the dried, isolated product analyzed by the hereinabove described HPLC method. The results are summarized in the table below.

### Example 5

One gram of PIE and 1 ml or dimethyl formamide (DMF) are charged to a test tube. The test tube is agitated by hand in a bath maintained at 45° C. All of the PIE does not dissolve. Three 1 ml aliquots of DMF are added to the test tube (total 4 ml). All of the PIE does not dissolve showing that DMF is not a high capacity solvent.

### Example 6

Fifty grams of PIE, 250 ml of DMF, and 50 g Pd/C catalyst (Johnson Matthey 87 L) were charged to a laboratory autoclave. The autoclave was closed, charged with H₂, and heated to 50° C. The H₂ pressure was adjusted to 3 atm. The contents of the autoclave were maintained at 50° C. under 3 atm H₂ for 72 hours.

Heating was ceased, the pressure released and the product worked-up by a procedure analogous to that used in Example 2. Analysis showed that ˜68.5% of PIE had been converted to pioglitazone containing about 3.5% impurities (HPLC). About 26.5% of the PIE remained unreacted.

What is claimed:

1. Pioglitazone containing less than about 0.14% area by HPLC of the impurity having RRT 0.64.

2. The pioglitazone of claim 1 containing less than about 0.02 area-% by HPLC of the impurity at RRT 0.64.

3. A method of making the pioglitazone of claim 1, comprising the steps of:
a) providing a solution of PIE in a high capacity solvent;
b) combining the solution with a supported metal hydrogenation catalyst in a reactor, wherein the supported metal hydrogenation catalyst comprises a metal selected from the group consisting of platinum, palladium, ruthenium, rhodium, osmium, and iridium;
c) heating the combination to a temperature of about 40° C. to about 100° C.;
d) separating the supported metal catalyst from the solution;
e) combining the solution with a crystallization solvent that selected from the group consisting of acetone and a lower aliphatic alcohol, and
f) recovering the solid pioglitazone formed.

4. The method of claim 3 wherein the high capacity solvent is formic acid.

5. The method of claim 3 wherein the combination of the solution and crystallization solvent is cooled to about 15° C. or below prior to the isolation step.

6. The method of claim 3 wherein the combination in step c) is heated to about 80° C.

7. The method of claim 3 wherein the crystallization solvent in step e) is ethanol.

8. The method of claim 3, further comprising, prior to step e), concentrating the solution from which catalyst has been separated.

9. The method of claim 3, wherein the pioglitazone contains about 0.02% area by HPLC.

10. Pharmaceutical compositions comprising the pioglitazone of either of claims 1 or 2, and at least one pharmaceutically acceptable excipient.
